(19) United States
(12) Patent Application Publication
(10) Pub. No.: US 2024/0156081 A1
(43) Pub. Date: May 16, 2024

(54) VENO-ARTERIAL VENOUS CROSS-CIRCULATION FOR EXTRACORPOREAL ORGAN SUPPORT

(71) Applicant: VANDERBILT UNIVERSITY, Nashville, TN (US)

(72) Inventors: Wei Kelly WU, Nashville, TN (US); Matthew BACCHETTA, Nashville, TN (US); Sophoclis ALEXOPOULOS, Nashville, TN (US); Rei UKITA, Nashville, TN (US); Andrew TUMEN, Louiseville, KY (US); John W. STOKES, Nashville, TN (US)

(21) Appl. No.: 18/284,060

(22) PCT Filed: Mar. 24, 2022
(86) PCT No.: PCT/US2022/021802

§ 371 Date: Sep. 25, 2023

Related U.S. Application Data
(60) Provisional application No. 63/165,773, filed on Mar. 25, 2021.

(51) Int. Cl. A01N 1/02; A61M 60/109; A61M 60/38; A61M 60/80

(52) U.S. Cl. CPC A01N 1/0247; A61M 60/109; A61M 60/38; A61M 60/80

(57) ABSTRACT

A system for extracorporeal organ support can include an organ chamber and a cross-circulation circuit. The organ camber can be configured to hold an extracorporeal organ. The cross-circulation circuit be configured to connect the extracorporeal organ and a host organism to maintain the extracorporeal organ by perfusing veno-arterial-venous (V-AV) blood through the extracorporeal organ and the host organism, wherein physiologic stability of the host organism is maintained.

### CROSS-REFERENCE TO RELATED APPLICATIONS

This application claims the benefit of U.S. Provisional Application No. 63/165,773, entitled “VENO-ARTERIAL VENOUS CROSS-CIRCULATION FOR EXTRACORPOREAL ORGAN SUPPORT,” filed 25 Mar. 2021. The entirety of this provisional application is hereby incorporated by reference for all purposes.

### TECHNICAL FIELD

The present disclosure relates generally to extracorporeal organ support and, more specifically, to veno-arterial venous (V-AV) cross-circulation for extracorporeal organ support while maintaining physical stability of a host.

### BACKGROUND

Cirrhosis of the liver is associated with a large global health burden. The only curative treatment for cirrhosis is liver transplantation; however, cirrhosis patients often linger on the liver transplant waiting list, risking mortality, due to a scarcity of suitable donor organs. Transplant waiting lists exist for all organs, with some waiting lists being shorter and many waiting lists being significantly longer than the liver transplant waiting list. The lack of viable donor organs for transplantation is enhanced due to significant bottlenecks arising from insufficient organ preservation and recovery strategies when a donor organ does become available.

### SUMMARY

The unmet need for improved salvage of organs can be met through improved extracorporeal organ support. By establishing an extracorporeal organ support mechanism that provides veno-arterial venous (V-AV) cross-circulation to maintain physiologic stability, the need for improved salvage can be met. The V-AV cross-circulation circuit employs an arterial limb and a venous limb to ensure that both the host and the extracorporeal organ retain physiologic stability.

In an aspect, the present disclosure can include a system for helping to maintain physiologic stability of the host organism and the extracorporeal organ. The system comprises an organ chamber configured to hold the extracorporeal organ and a cross-circulation circuit. The cross-circulation circuit is configured to connect the extracorporeal organ and the host organism to maintain the extracorporeal organ by perfusing veno-arterial-venous (V-AV) blood through the extracorporeal organ and the host organism. The physiologic stability of the host organism is also maintained.

In another aspect, the present disclosure can include a method for helping to maintain physiologic stability of the host organism and the extracorporeal organ. The method includes the following steps. Maintaining viability of the extracorporeal organ located in an organ chamber. Cannulating at least one vein and one artery in the extracorporeal organ. Cannulating at least two veins of the host organism and an artery of the host organism. Establishing a cross-circulation circuit by connecting one of the at least two veins of the host organism to be in fluid communication with the artery of the host organism and to be in fluid communication with the at least one artery in the extracorporeal organ, and the at least one vein of the extracorporeal organ to be in fluid communication with another of the at least two veins of the host organism. Perfusing V-AV blood through the extracorporeal organ and the host organism, where physiologic stability of the host organism is maintained.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1 and 2 are schematic diagrams of an example of the perfusion system;

FIGS. 3 and 4 are schematic diagrams of another example of the perfusion system;

FIGS. 5-7 are process flow diagrams illustrating methods for establishing a cross-circulation circuit of the perfusion system and using the perfusion system;

FIG. 8 is a schematic diagram showing an experimental perfusion system connecting an external donor liver to a host;

FIGS. 9 and 10 are plots showing experimental results using the experimental perfusion system of FIG. 8;

FIG. 11 is a schematic diagram showing another experimental system connecting another external donor liver to another host;

FIG. 12 includes photographs of portions of extracorporeal livers as part of the experimental perfusion system of FIG. 11;

FIGS. 13 and 14 are plots showing experimental results using the experimental perfusion system of FIG. 11; and

FIG. 15 is histological results using the experimental perfusion system of FIG. 11.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “circuit” refers to a complete and closed path through which a liquid, such as blood, a man-made perfusate, or the like, can flow.

As used herein, the term “perfusion circuit” refers to a path that blood, a man-made perfusate, or the like, flows through to supply oxygen and nutrients to one or more organs or tissue.

As used herein, the term “machine perfusion” refers to a technique used in organ transplant as an alternative to traditional cold storage, where a perfusate is pumped out of a reservoir (or host organism), oxygenated, and then pumped into an extracorporeal organ to help maintain organ viability for transplant. Described herein is a type of machine perfusion that can employ a veno-arterial venous (V-AV) cross-circulation circuit to maintain organ viability, but can also ensure that the host organism maintains physiologic stability.

As used herein, the term “veno-arterial venous (V-AV)” refers to a type of extracorporeal membrane oxygenation where blood is pumped out of a body of a host organism through one vein (e.g., an internal jugular vein) and oxygenated before being returned back into the body of the host organism through both an artery of the host organism and through a vein of the host organism. For example, when the organ is a liver, blood can be pumped out of an internal jugular vein of the host and oxygenated before part of the oxygenated blood is pumped to a common femoral artery of the host and the other part of the oxygenated blood is pumped into a hepatic artery and portal vein of the liver to perfuse the liver before the perfused blood is returned to a contralateral internal jugular vein of the host. In another example, the blood can be returned to the same internal jugular vein it was pumped from using a dual lumen cannula.

As used herein, the term “V-AV cross-circulation circuit” refers to a perfusion circuit that circulates V-AV blood between an extracorporeal organ and a host while maintaining physiologic stability of both.

As used herein, the term “V-AV blood” refers to the blood that flows through a V-AV cross-circulation circuit. V-AV blood includes blood pumped out of a vein of the host, oxygenated blood pumped into an artery of the host and the artery and/or vein of the extracorporeal organ, and the de-oxygenated blood that has perfused the organ and is then returned to a vein of the host.

As used herein, the term “normothermic” refers to an environmental temperature that does not cause increased or decreased activity of cells of a body. For a human body the peak normothermic temperature range is between approximately 36 degrees Celsius and 38 degrees Celsius.

As used herein, the term “physiologic stability” refers to a dynamic range of physiological parameters that characterize normal function of an organism and/or one or more organs that make up the organism, that are not suffering from disease or injury. Physiological parameters can include, but are not limited to, oxygen saturation, pressure, temperature, and pH level.

As used herein, the term “host organism” refers to an organism acting as a host for an extracorporeal organ.

As used herein, the term “organism” can refer to any warm-blooded organism including, but not limited to, a human being, a pig, a rat, a mouse, a dog, a car, a goat, a sheep, a horse, a monkey, an ape, a rabbit, a cow, etc.

As used herein, the term “host” can refer to an organism that acts as the support system for an extracorporeal organ, such as a transplant recipient.

As used herein, the term “extracorporeal organ” refers to an organ situated outside the body of an organism (e.g., an organ provided by an organ donor, a lab grown organ, or an organ detached (voluntarily or involuntarily) from the host organism). An extracorporeal organ can include, but is not limited to, an internal organ (e.g., heart, liver, lungs, kidney, pancreas, small intestine, gut, etc.), an external organ (e.g., skin), tissue, a bioengineered graft, a xenogenic organ graft or a limb (e.g., arm, leg, hand, foot, etc.).

As used herein “extracorporeal” refers to something situated or occurring outside of the body of an organism.

As used herein, the term “contralateral” refers to something relating to or denoting the side of the body opposite to that on which a particular structure or condition occurs.

### II. Overview

Traditionally, donor organs have been preserved through cold storage, which has significant limitations in duration and quality of preservation. Normothermic machine perfusion (NMP) has emerged as an alternative organ preservation method with desirable features like continuous delivery of oxygen and nutrients, flushing of waste products, and opportunity to monitor graft viability ex-vivo prior to transplantation. NMP also offers opportunities as a research platform for the study of ex-vivo therapies, such as defatting protocols, immunomodulation, RNA interference, and anti-inflammatory agents. However, despite the provision of oxygen and circulatory support to donor organs, isolated single-organ support systems lack the ability to duplicate the myriad hemodynamic, hematologic, metabolic, endocrine, and biochemical process that maintain homeostasis in vivo. A system that can recapitulate a normal physiologic milieu for the extracorporeal organ will better enable organ rescue, recovery, and investigation of advanced interventions.

Although systems have been designed to better enabled organ rescue, recovery, and investigation of advanced interventions, these systems have ignored the need to maintain physiologic stability of the host organism as well. The present disclosure describes a veno-arterial venous (V-AV) cross-circulation platform with the potential to offer total physiological support to a donor organ and host organism within a homeostatic biosystem. A cross-circulation platform does not need to duplicate the myriad hemodynamic, hematologic, metabolic, endocrine, and biochemical process that maintain homeostasis in vivo. Instead, the V-AV cross-circulation platform connects a host organism having the homeostatic biosystem required by the donor organ directly to the extracorporeal donor organ to maintain physiological support using the host organism's body fluids. Contrary to traditional solutions, the V-AV cross-circulation platform adds the arterial limb to direct oxygenated blood to an artery of the host organism as a method of improving maintenance of physiological stability in the host organism. The V-AV cross-circulation platform improves extracorporeal organ viability for research and transplant purposes while ensuring the host remains in homeostasis. Additionally, the cross-circulation circuit can permit more careful monitoring, functional testing, assessment, and therapy of the harvested organ. This would in turn allow earlier detection and potential repair of defects in the harvested organ, further reducing the likelihood of post-transplant organ failure. The ability to perform and assess simple repairs on the organ would also allow many organs with minor defects to be saved, whereas current transplantation techniques often require such organs to be discarded.

### III. Systems

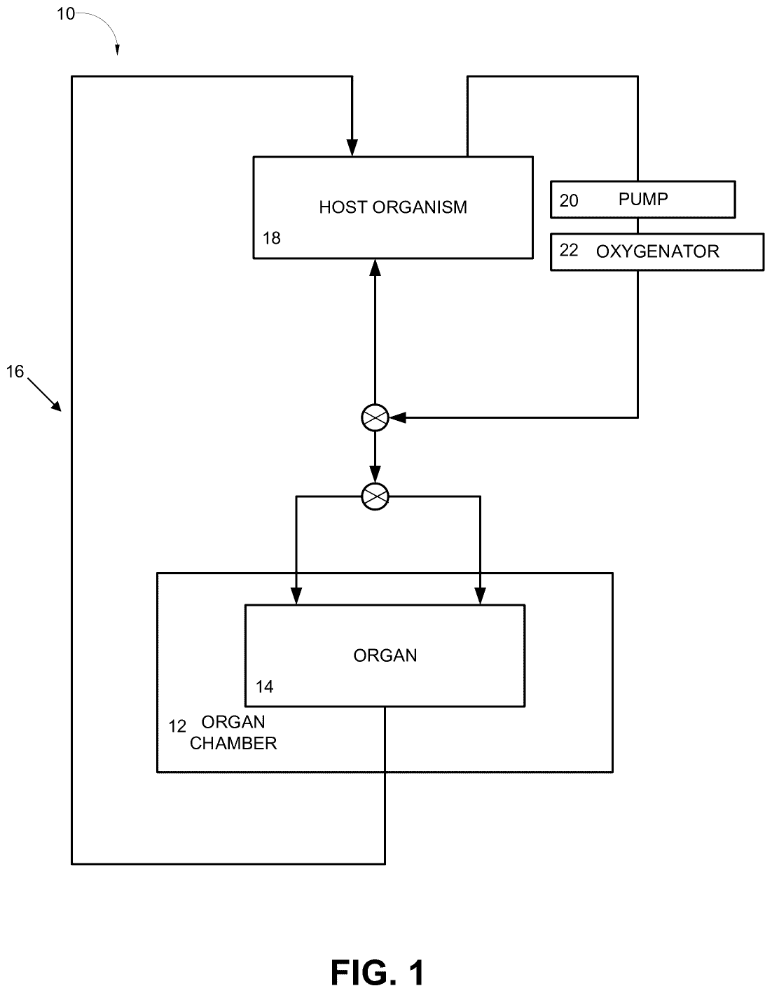

An aspect of the present disclosure can include a system 10 (FIG. 1) that can employ machine perfusion with a veno-arterial venous (V-AV) cross-circulation circuit that leverages the intrinsic physiologic milieu provided by the host organism to improve hemodynamic stability of both the host organism and the extracorporeal organ. The system 10 maintains the quality and function of the extracorporeal organ and provides hemodynamic support and improved physiologic homeostasis for the host organism through the utilization of the V-AV cross-circulation circuit, where the arterial loop provides this support. Additionally, the system 10 can provide support for the extracorporeal organ and/or the host organism for an extended duration compared to traditional cold storage techniques.

The system 10 includes an organ chamber 12 configured to hold an extracorporeal organ 14 and a cross-circulation circuit 16. The cross-circulation circuit 16 is configured to connect the extracorporeal organ 14 and a host organism 18. The cross-circulation circuit 16 can maintain the extracorporeal organ 14 by perfusing blood through the extracorporeal organ and back to the host organism 18 in a V-AV circuit configuration. The blood, which may be referred to as V-AV blood, can flow through the cross-circulation circuit 16 from a vein of the host organism 18 to an artery of the host organism and to an artery and/or vein of the extracorporeal organ 14. Blood that has perfused the extracorporeal organ 14 can then flow out of a vein of the extracorporeal organ and back to the host organism 18 through the venous system, through another vein or the same vain, through the cross-circulation circuit 16.

V-AV blood refers to the path the blood takes through the extracorporeal organ 14, cross-circulation circuit 16, and the host organism 18; where blood from a vein of the host organism can enter the cross-circulation circuit from a vein, be oxygenated in the cross-circulation circuit, and then (1) returned to the body of the host organism through an artery and (2) perfused through the extracorporeal organ before it can be returned to the body of the host organism 18 through the venous system of the host organism 18 using another vein or the same vein. The cross-circulation circuit 16 can connect a vein of the host organism 18 with an artery of the host organism and with at least one of an artery or a vein of the extracorporeal organ 14 using tubing attached to cannulations at the veins and arteries. Blood can be moved through the system 10 by using a pump 20 in line with the tubing in the cross-circulation circuit 16. The pump 20 can use negative pressure to pull blood from the host organism 18 into the cross-circulation circuit 16 (e.g., a tube). The pump 20 can then use positive pressure to push the blood through an oxygenator 22 and into the extracorporeal organ 14 and back to the host organism 18 through the tubing attached to the cannulations of the at least one vein or artery of the extracorporeal organ and the artery of the host organism. The oxygenator 22 adds oxygen to the blood before the blood is pumped into the extracorporeal organ 14 and back to the host organism 18. The addition of oxygenated blood to the extracorporeal organ 14 and the host organism 18 helps to sustain physiologic stability of both organ and organism. The cross-circulation system 16 can also separately connect the extracorporeal organ 14 (e.g., a vein) and the host organism 18 (e.g., another vein) for the blood to flow back to the host organism once it has perfused the extracorporeal organ. The extracorporeal organ 14 can be, but is not limited to, a liver, a lung, a kidney, a heart, a limb, skin, or a tissue substrate. When the extracorporeal organ 14 is a lung the oxygenator 22 is not necessary in system 10.

In one example, the cross-circulation circuit 16 can be configured to connect at least one vein of the host organism 18 with at least one artery of the host organism and an artery and/or vein of the extracorporeal organ 14, and also to connect at least one vein of the extracorporeal organ with another at least one vein of the host organism. The pump 20 can be configured to pump the V-AV blood from the at least one vein of the host organism 18 through the oxygenator 22 to the at least one artery of the host organism and to the artery and/or vein of the extracorporeal organ. When the extracorporeal organ 14 is a liver, the liver can be connected to the host organism 18 by the cross-circulation circuit 16 and V-AV blood can be perfused through the host organism, the cross-circulation circuit, and the liver. A portal vein of the liver, a hepatic artery of the liver, and an infrahepatic inferior vena cava in the liver can be cannulated to allow perfusion of V-AV blood through the cross-circulation circuit 16, the liver 14, and the host organism 18. The infrahepatic inferior vena cava can be connected to a peripheral or central vein of the host organism 18 by the cross-circulation circuit 16. Another peripheral or central vein of the host organism 18 can be connected with the portal vein and the hepatic artery of the liver 14 through the pump 20 and the oxygenator 22. The other peripheral or central vein of the host organism 18 can also be connected with the peripheral or central artery of the host organism. For example, the peripheral or central vein of the host organism 18 and the other peripheral or central vein of the host organism can be at least one of the right internal jugular vein or the left internal jugular vein. A femoral vein of the host organism 18 may also be used. The peripheral or central artery of the host organism 18 can be one of the common femoral artery, the carotid artery, the subclavian artery, or the aorta.

In one aspect, the right internal jugular vein of the host organism 18 can be connected to the common femoral artery of the host organism and to the portal vein and the hepatic artery of the liver 14 by the cross-circulation circuit 16 via the pump 20 and an oxygenator 22. A flow regulator in the cross-circulation circuit 16 can be configured to split the flow of oxygenated V-AV blood to flow partially to the common femoral artery of the host organism 18 and partially to the liver 14. A second flow regulator in the cross-circulation circuit 16 can also split the oxygenated V-AV blood flow towards the liver to flow partially into the hepatic artery and partially into the portal vein. The cross-circulation circuit 16 can also be configured to connect the infrahepatic inferior vena cava in the liver 14 to the left internal jugular vein of the host organism 18 such that the V-AV blood that has perfused the liver flows from the infrahepatic inferior vena cava to the left internal jugular vein to be returned to the host organism and pumped back through the heart.

Other example configurations of the connections of the cross-circulation circuit 16 with the host organism 18 and the extracorporeal organ 14 would be obvious to a person skilled in the art if the extracorporeal organ is an organ other than the liver or if one of the veins or arteries discussed above cannot be utilized due to disease or damage.

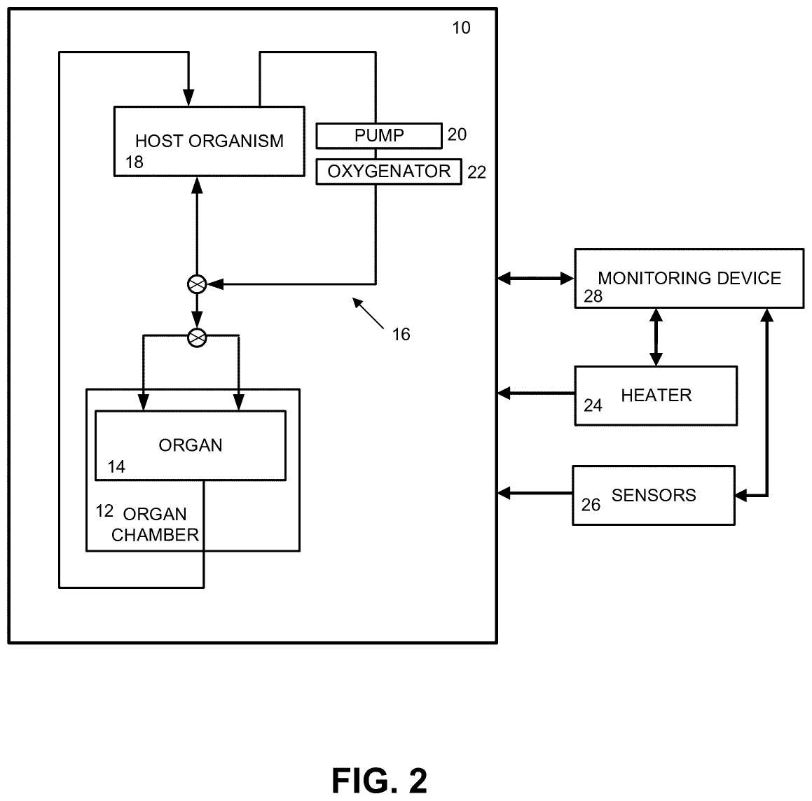

Referring now to FIG. 2, the system 10 can also employ one or more of a heater 24, a plurality of sensors 26, and a monitoring device 28. The heater 24, the plurality of sensors 26, and the monitoring device 28 can all be utilized to help maintain the physiological stability of the extracorporeal organ 14 and the host organism 18 when the system 10 is in use.

The heater 24 can keep the extracorporeal organ 14, the host organism 18, and the cross-circulation circuit 16 at a constant temperature from 10 degrees Celsius to 50 degrees Celsius. The constant temperature can also be a normothermic temperature. Maintaining a constant temperature of the system 10 improves viability of the extracorporeal organ 14 and the host organism 18 because temperature changes of even 2 degrees Celsius can cause significant damage to the extracorporeal organ and the host organism. The plurality of sensors 26 can be configured to detect changes in at least one parameter of the cross-circulation circuit 16, the extracorporeal organ 14, and/or the host organism 18. The at least one parameter includes at least one of a blood flow rate, a cross circulation blood flow, an organ inflow pressure, an organ outflow pressure, a host hemodynamics value, a circuit temperature, and a host temperature. The plurality of sensors 26 can be configured to be positioned at locations throughout the cross-circulation circuit 16, the organ chamber 12, the extracorporeal organ 14, and the host organism 18. The plurality of sensors 26 can include, but are not limited to, temperature sensors, pressure sensors, flow rate sensors, and oximeters. The monitoring device 28 can be configured to monitor changes detected by the plurality of sensors 26 and can be configured to alert a medical professional when the changes are outside a predetermined threshold. The predetermined threshold(s) can be specific to the type of extracorporeal organ 14 and/or the host organism 18 in the system 10 or the predetermined threshold(s) can be general based on previous research. The monitoring device 28 can also be configured to control at least one of the pump 20, the oxygenator 22, and the heater 24 in response to the detected changes in the at least one parameter outside of the pre-determined threshold to return the at least one parameter to within the predetermined threshold.

To maintain a constant and physiologically stable environment the at least one parameter should be maintained within the predetermined threshold. Pressures of the extracorporeal organ 14 and the host organism 18 can be maintained by keeping the extracorporeal organ at a certain height relative to the host organism. The organ chamber 12 can be held (e.g., by a stand, cart, etc.) at a variable height with respect to the height of the host organism 18. The variable height of the organ chamber 12 can be used to keep the extracorporeal organ 14 and the host organism 18 at a near physiological pressure. For example, if the extracorporeal organ 14 is a liver to the target portal venous pressure can be less than 15 mmHg and the target hepatic venous pressure gradient (HVPG) can be less than 10 mmHg. The height of the extracorporeal organ 14 (e.g., the liver) can be adjusted with respect to the host organism 18 to meet these target pressures. The heater 24 also helps to maintain a physiologically stable environment for the extracorporeal organ 14 and the host organism 18 by heating the system between 10 degrees Celsius and 50 degrees Celsius. A flow regulator can be configured to control the rate of blood pumped into the at least one artery of the host organism 18 and the rate of blood pumped into the at least one vein or artery of the extracorporeal organ. The oxygenator 22 can be configured to maintain a physiologic level of blood oxygen saturation in the blood perfusing the extracorporeal organ and the host organism 18 by injecting a gas mixture including oxygen into the blood as it passes through the oxygenator. A physiologic level of blood oxygen saturation can be between 60% and 100%, 80% and 100%, 90% to 100%, or 95% to 100% depending on if venous oxygen saturation or arterial oxygen saturation is measured. Venous oxygen saturation levels can be lower than arterial oxygen saturation levels without ischemia occurring.

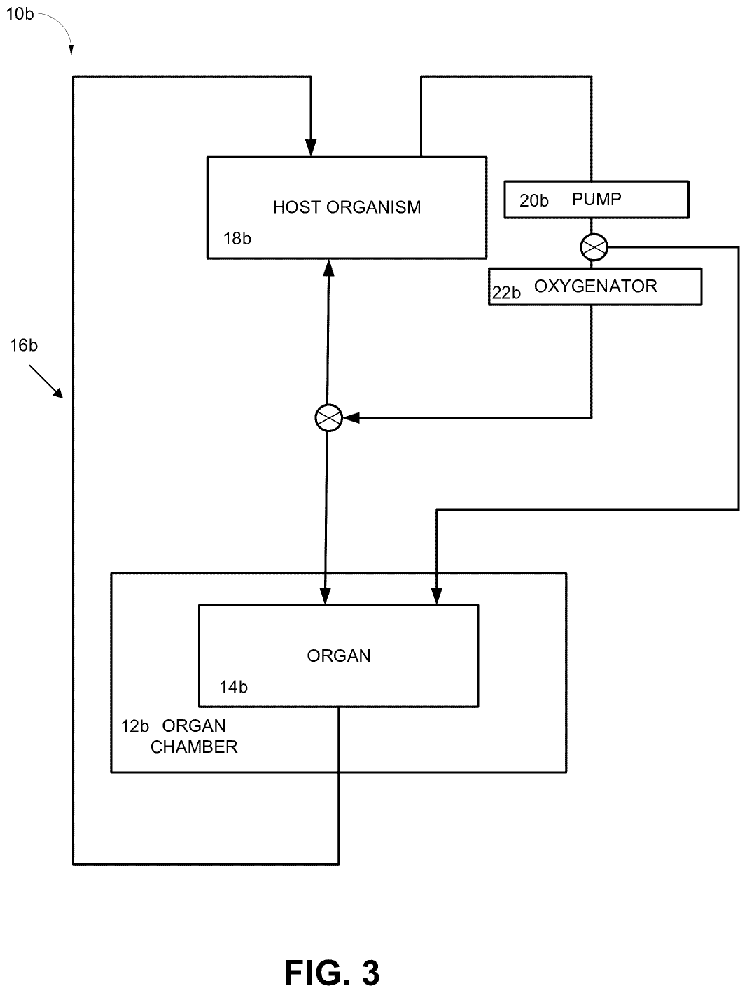

Referring now to FIG. 3, another example configuration of the system 10b is shown. The system 10b includes an organ chamber 12b configured to hold an extracorporeal organ 14b and a cross-circulation circuit 16b. The cross-circulation circuit 16b is configured to connect the extracorporeal organ 14b and a host organism 18b. The cross-circulation circuit 16b can maintain the extracorporeal organ 14b by perfusing blood through the extracorporeal organ and back to the host organism 18b in a V-AV circuit configuration. The blood, which may be referred to as V-AV blood, can flow through the cross-circulation circuit 16b from a vein of the host organism 18b to an artery of the host organism and to an artery and/or vein of the extracorporeal organ 14b. Blood that has perfused the extracorporeal organ 14b can then flow out of a vein of the extracorporeal organ and back to another vein of the host organism 18b through the cross-circulation circuit 16b.

V-AV blood refers to the path the blood takes through the extracorporeal organ 14b, cross-circulation circuit 16b, and the host organism 18b; where blood from a vein of the host organism can enter the cross-circulation circuit from a vein, be oxygenated in the cross-circulation circuit, and then (1) returned to the body of the host organism through an artery and (2) perfused through the extracorporeal organ before it can be returned to the body of the host organism through another vein. Additionally, blood pumped from a vein of the host organism 18b can also directly travel to the extracorporeal organ 14b without being oxygenated in the cross-circulation circuit 16b. The cross-circulation circuit 16b can connect a vein of the host organism 18b with an artery of the host organism and with at least one of an artery or a vein of the extracorporeal organ 14b using tubing attached to cannulations at the veins and arteries. Blood can be moved through the system 10b by using a pump 20b in line with the tubing in the cross-circulation circuit 16b. The pump 20b can use negative pressure to pull blood from the host organism 18b into the cross-circulation circuit 16b (e.g., a tube). The pump 20b can then use positive pressure to push the blood through an oxygenator 22b and into the extracorporeal organ 14b and back to the host organism 18b through the tubing attached to the cannulations of the at least one vein or artery of the extracorporeal organ and the artery of the host organism. The oxygenator 22b adds oxygen to the blood before the blood is pumped into the extracorporeal organ 14b and back to the host organism 18b. The addition of oxygenated blood to the extracorporeal organ 14b and the host organism 18b helps to sustain physiologic stability of both organ and organism. The pump 20 can also use negative pressure to pull blood from the host organism and push the into the extracorporeal organ 14b, without passing through the oxygenator. In this way the extracorporeal organ 14b receives partially oxygenated blood, which may be akin to what happens in the human body, where portal vein blood is only partially oxygenated. The cross-circulation system 16b can also separately connect the extracorporeal organ 14b (e.g., a vein) and the host organism 18b (e.g., another vein) for the blood to flow back to the host organism once it has perfused the extracorporeal organ. Each example configuration described with respect to the system 10 of FIG. 1 can also work with the configuration of system 10b shown in FIG. 3.

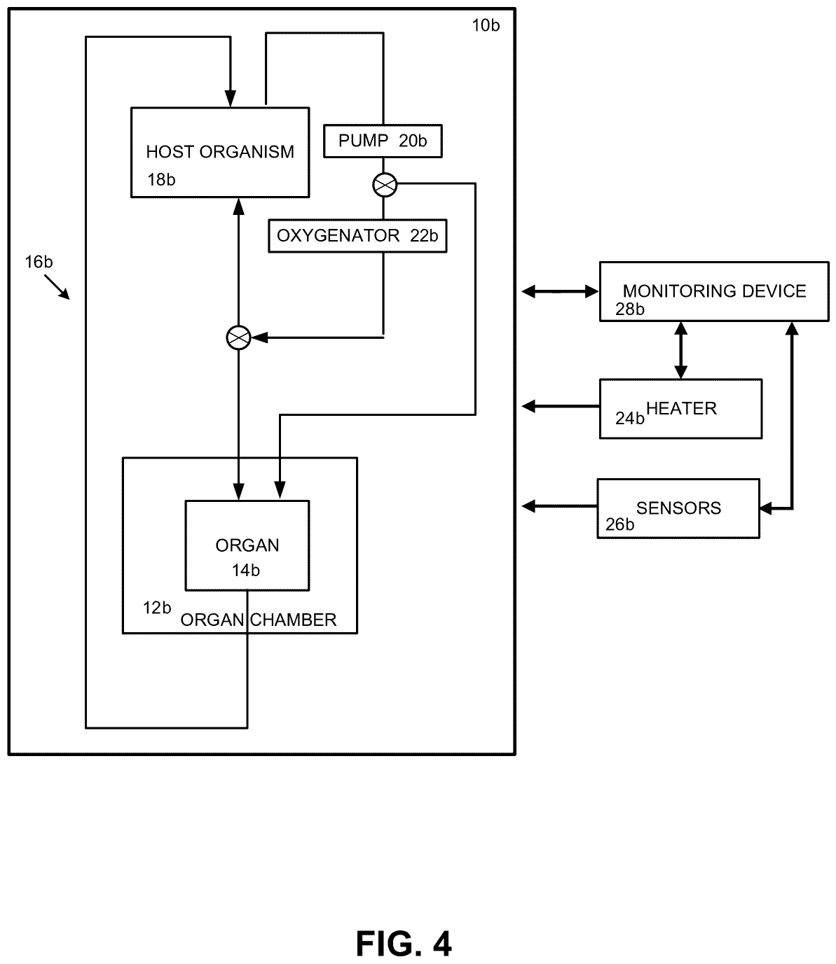

Referring now to FIG. 4, the system 10b can also employ one or more of a heater 24b, a plurality of sensors 26b, and a monitoring device 28b. The heater 24b, the plurality of sensors 26b, and the monitoring device 28b can all be utilized to help maintain the physiological stability of the extracorporeal organ 14b and the host organism 18b when the system 10b shown in FIG. 3 is in use and the blood entering the extracorporeal organ is only partially oxygenated.

### IV. Methods

Another aspect of the present disclosure can include methods 90, 100, and 110 for maintaining physiologic stability of a host organism and an extracorporeal organ. The methods 90, 100, and 110 can be executed using the system 10 shown in FIGS. 1 and 2 or the system 10b shown in FIGS. 3 and 4. For purposes of simplicity, the methods 90, 100, and 110 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 90, 100, and 110, nor are methods 90, 100, and 110 limited to the illustrated aspects.

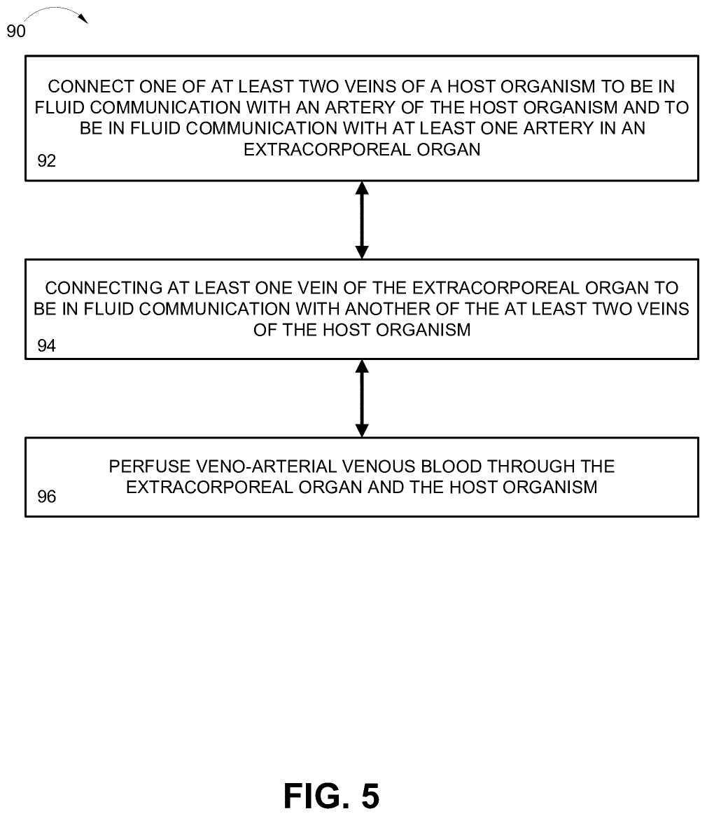

Referring now to FIG. 5, illustrated is a method 90 for establishing a cross-circulation circuit configured to perfuse V-AV blood between a host organism and an extracorporeal organ. At 92, one of at least two veins of the host organism can be connected with an artery of the host organism and with at least one artery in an extracorporeal organ, such that the vein and the artery of the host organism are in fluid communication through the circuit and the vein and the at least one artery of the extracorporeal organ are in fluid communication through the circuit. A pump and an oxygenator can be part of the connection such that the pump can be configured to pump blood from the one of the at least two veins of the host organism through the oxygenator so that oxygenated blood can be pumped into the artery of the host organism and into the at least one artery of the extracorporeal organ. Additionally or alternatively, the pump may be configured to pump blood from the one of the at least two veins of the host organism directly into the at least one artery of the extracorporeal organ without being oxygenated. In this way the extracorporeal organ may receive partially oxygenated blood in the cross-circulation circuit, which may be more akin to what happens in the human body because portal vein blood is naturally only partly oxygenated. At 94, at least one vein of the extracorporeal organ can be connected to another of the at least two veins of the host organism, such that the vein of the organ is in fluid communication with the vein of the host organism through the circuit. The at least two veins of the host organism can be an internal jugular vein and a contralateral internal jugular vein, then the at least one vein of the extracorporeal organ can be connected with an internal jugular vein of the host organism and the artery of the host organism can be connected with the contralateral internal jugular vein of the host organism. At 96, V-AV blood can be perfused through the extracorporeal organ and the host organism to maintain physiologic stability of both the extracorporeal organ and the host organism. To maintain physiologic stability the extracorporeal organ, the host organism, and the cross-circulation circuit can also be maintained at a constant temperature from 10 degrees Celsius to 50 degrees Celsius. Additionally, the flow of V-AV blood from the one of the at least two veins of the host organism to the artery of the host organism and to the at least one artery of the extracorporeal organ can be regulated to further facilitate maintaining physiologic stability.

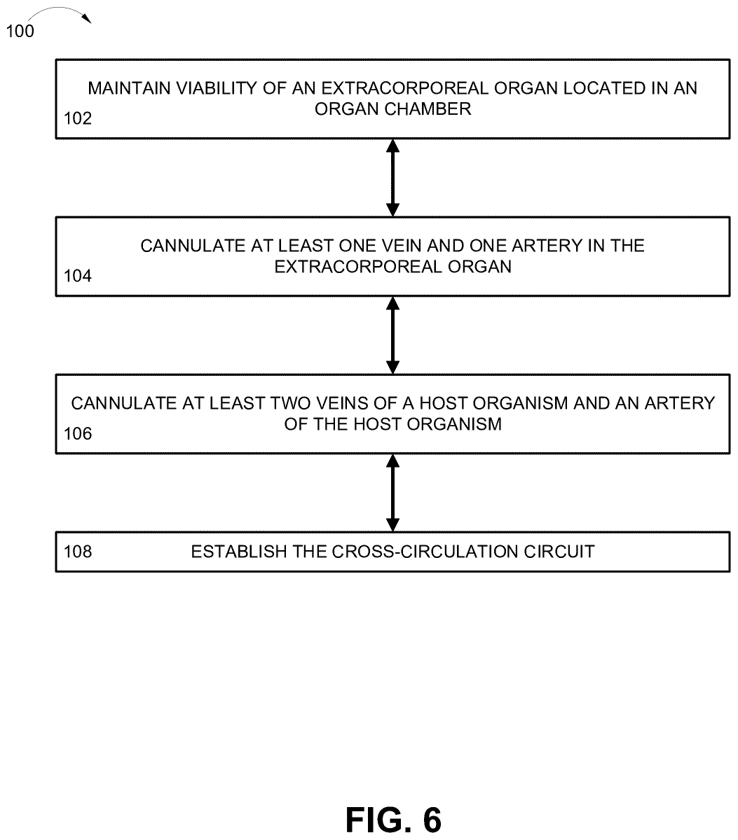

Referring now to FIG. 6, illustrated is a method 100 for preparing the host organism and the extracorporeal organ for connection to the cross-circulation circuit. At 102, viability of an extracorporeal organ located in an organ chamber is maintained. Viability can be maintained with traditional cold storage techniques or normothermic machine perfusion techniques. At 104, the at least one vein and one artery in the extracorporeal organ can be cannulated to allow for connection of the cross-circulation circuit to the extracorporeal organ. At 106, the at least two veins of the host organism and the artery of the host organism can be cannulated to allow for connection of the cross-circulation circuit to the host organism. The at least two veins of the host organism that can be cannulated can be an internal jugular vein and a contralateral internal jugular vein of the host organism (e.g., right IJV and left IJV). In another example, only one vein of the host organism may be cannulated if it is cannulated with a dual lumen cannula and the vein can be one of an internal jugular vein or a femoral vein. The artery of the host organism can be, but is not limited to, one of a common femoral artery, a carotid artery, a subclavian artery, or an aorta. At 108, the cross-circulation circuit can be established by connecting the host organism and the extracorporeal organism such that they are in fluid communication using the method 90 described above.

Establishing the cross-circulation circuit can include connecting the at least one vein of the extracorporeal organ with the internal jugular vein of the host organism and connecting the artery of the host organism with the contralateral internal jugular vein of the host organism. In one example, the extracorporeal organ can be a liver and cannulating the at least one vein and one artery in the extracorporeal organ can include cannulating the liver's hepatic artery, infrahepatic inferior vena cava, and the portal vein. The cross-circulation circuit can be established by connecting the internal jugular vein to the artery of the host organism and to the portal vein and hepatic artery of the liver, and connecting the infrahepatic inferior vena cava to the contralateral internal jugular vein of the host organism. Blood can flow from the internal jugular vein of the host organism to the artery of the host organism and to the hepatic artery and the portal vein of the liver through the circuit. The blood can also flow from the liver through the infrahepatic inferior vena cava to the contralateral internal jugular vein of the host organism.

The method 100 can include additional steps, not shown, to facilitate maintaining physiologic stability of the host organism and the extracorporeal organ. A plurality of sensors can be positioned throughout the system and can detect changes in at least one parameter of the cross-circulation circuit, the organ, and the host organism. A monitoring device, which can include a processor and a non-transitory memory, can monitor the change in the at least one parameter of the cross-circulation circuit, the organ, and the host organism. The at least one parameter can include at least one of blood flow rates, cross circulation blood flow, organ inflow pressure, organ outflow pressure, host hemodynamics, circuit temperature, and host temperature. The monitoring device can also include a display, where the display can alert (e.g., by a visual, auditory, or tactile alert) a medical professional when changes to the at least one parameter are outside a pre-determined threshold.

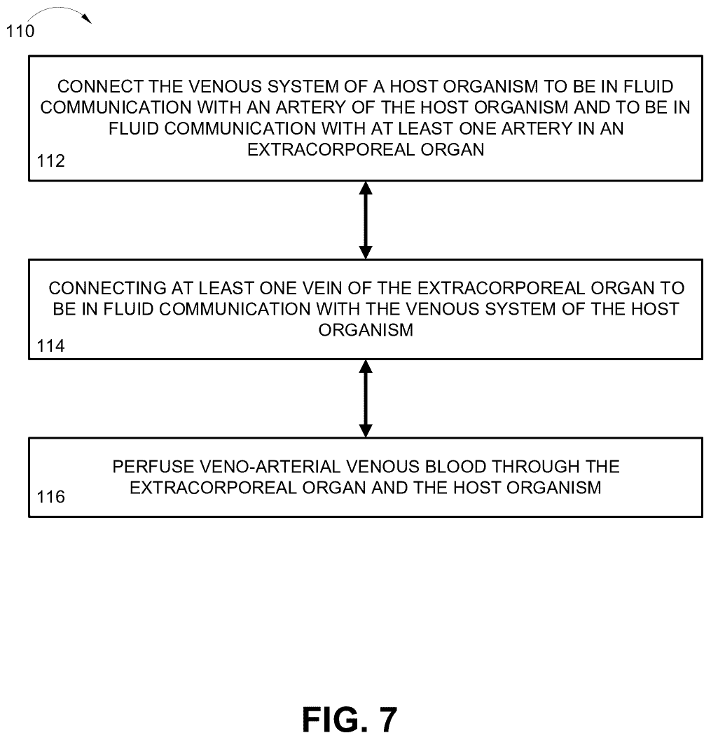

In another example, shown in FIG. 7, illustrated is a method 110 for establishing a cross-circulation circuit configured to perfuse V-AV blood between a host organism and an extracorporeal organ. At 112, the venous system of the host organism can be connected with an artery of the host organism and with at least one artery in an extracorporeal organ, such that the venous system and the artery of the host organism are in fluid communication through the circuit and the venous system and the at least one artery of the extracorporeal organ are in fluid communication through the circuit. A pump and an oxygenator can be part of the connection such that the pump can be configured to pump blood from a venous system of the host organism through the oxygenator so that oxygenated blood can be pumped into the artery of the host organism and into the at least one artery of the extracorporeal organ. Additionally or alternatively, the pump may be configured to pump blood from the venous system of the host organism directly into the at least one artery of the extracorporeal organ without being oxygenated. In this way the extracorporeal organ may receive partially oxygenated blood in the cross-circulation circuit, which may be more akin to what happens in the human body because portal vein blood is naturally only partly oxygenated. At 114, the venous system of the extracorporeal organ can be connected to such that a vein of the organ is in fluid communication with the venous system of the host organism through the circuit. The venous system of the host organism can be a femoral vein, an internal jugular vein, or a contralateral internal jugular vein. A single vein of the host organism can be used by cannulating the singular vein with a dual lumen cannula, then the singular vein of the extracorporeal organ can be connected with an internal jugular vein or femoral vein of the host organism and the artery of the host organism. In this example the single vein of the host organism can be cannulated with a dual lumen cannula of sufficient size for continuous blood flow through both lumens. At 116, V-AV blood can be perfused through the extracorporeal organ and the host organism to maintain physiologic stability of both the extracorporeal organ and the host organism. To maintain physiologic stability the extracorporeal organ, the host organism, and the cross-circulation circuit can also be maintained at a constant temperature from 10 degrees Celsius to 50 degrees Celsius. Additionally, the flow of V-AV blood from the venous system of the host organism to the artery of the host organism and to the at least one artery of the extracorporeal organ can be regulated to further facilitate maintaining physiologic stability.

### V. EXAMPLES

### Example 1

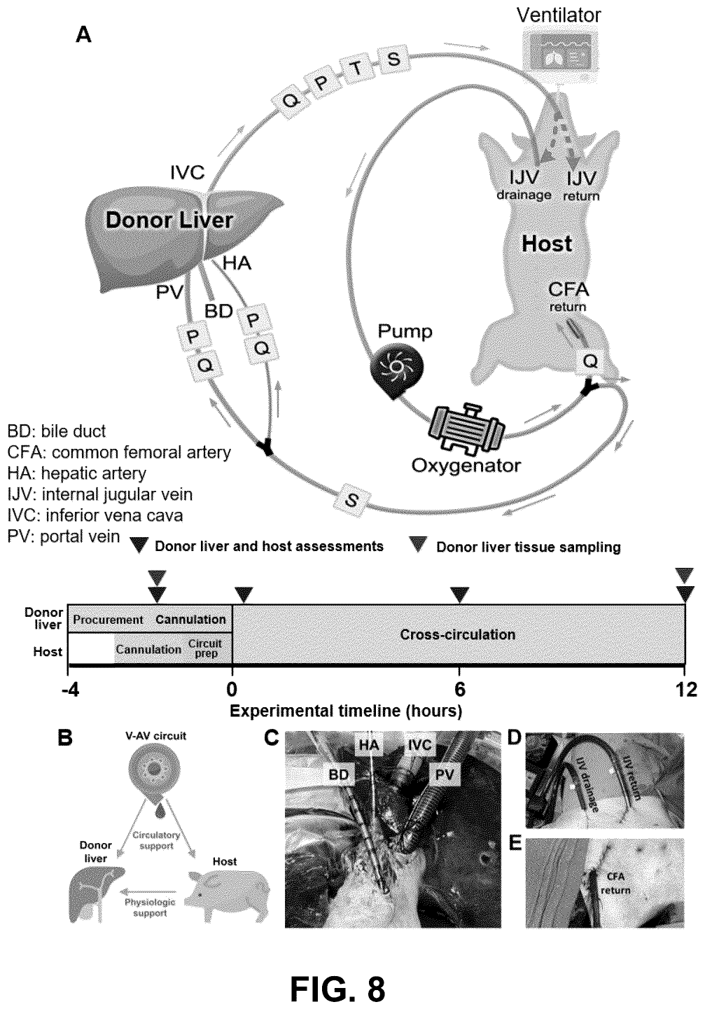

This example demonstrates that a swine cross-circulation platform (shown in FIG. 8, top) enables both extracorporeal donor liver preservation and host hemodynamic support. Based on this Experiment, the homeostatic normothermic extracorporeal support can be extended in duration from days to weeks, can be applied in a xenogeneic setting to unallocated donor livers, and has the potential to offer new opportunities for the assessment, recovery, and regeneration of human donor livers.

Given its ability to provide hemodynamic support, it is envisioned that this configuration of the cross-circulation platform may also address many of the challenges seen in combined heart-liver transplantation, where the heart is often transplanted first and followed by a period of pharmacologic or mechanical circulatory support as the graft recovers function. Cross-circulatory support of the extracorporeal liver graft, in addition to mechanical circulatory support of the heart transplant recipient, could support early cardiac graft function, maintain normothermic perfusion of the donor liver, minimize liver cold ischemic time, and optimize the recipient for sequential liver transplantation. Alternatively, potential transplant recipients could serve as cross-circulation ‘hosts’ to enable functional assessment and maintenance of donor organs before transplantation. The cross-circulation platform would thereby enable the assessment and recovery of high-risk donor livers without the concurrent stress of a surgical transplantation procedure. These livers would be transplanted into the host recipient upon meeting acceptable transplant criteria. Beyond clinical applications, liver cross-circulation creates novel opportunities for extracorporeal liver manipulation and optimization within a homeostatic bioreactor. The physiologic milieu of cross-circulation may be preferable to single-organ support systems for research and development of techniques and therapeutics that rely upon, or are affected by, interactions only present in a more complete biosystem. Future investigations using extended organ support could enable advanced interventions through chemical conditioning, immunomodulation, viral transfection, cell replacement, or other bioengineering approaches to improve organ function. It is envisioned a potentially broad application for this system as a translational research and basic science tool to develop technology that enables organ recovery, rehabilitation, and regeneration.

Materials and Methods

Study Design

This investigation was designed as a feasibility study (n=4) to assess the ability of the veno-arterial venous cross-circulation circuit V-AV XC system (FIG. 8 element A) to maintain the quality and function of explanted swine livers for 12 hours while simultaneously providing hemodynamic support and improved physiologic homeostasis in the swine host. 12 hours was deemed a sufficient duration to assess the feasibility of cross-circulation since normothermic explanted livers are susceptible to injury, dysregulation, and loss of synthetic function without adequate support. It is hypothesized that the V-AV XC system could provide physiologic support to both the explanted swine livers and the bioreactor host. This study was conducted with the minimum number of animals to demonstrate feasibility and reproducibility between livers and hosts, and across experimental time points.

Animals

Eight closed colony-bred male Yorkshire×Landrace swine (four donor-host pairs; Oak Hill Genetics) were utilized in this study. Animals were 3-5 months of age, with a weight range of 52-68 kg for animals used as liver donors, and a weight range of 55-88 kg for animals used as XC hosts. All studies complied with the relevant ethical regulations for animal testing and research. This study was approved by the Institutional Animal Care and Use Committee at Vanderbilt University Medical Center. All animal care and procedures were conducted in accordance with the US National Research Council of the National Academies Guide for the Care and Use of Laboratory Animals, Eighth Edition.

Donor Liver Procurement

Livers were procured from 4 healthy swine donors. Anesthetic induction was achieved with ketamine (2.2 mg/kg intramuscular [IM]), Telazol® (4.4 mg/kg IM), xylazine (2.2 mg/kg IM), and isoflurane (1-3% inhaled). Subjects were intubated and appropriate anesthetic monitors were placed. Inhaled isoflurane (1-3%) and intravenous (IV) fentanyl (0.03-0.1 mg/kg/hr) were used for anesthetic maintenance and analgesia. Prior to skin incision, animals were prepped and draped in standard sterile fashion and antibiotics were administered (cefazolin, 20 mg/kg; enrofloxacin, 5 mg/kg). Following midline laparotomy, mobilization of the liver, and standard dissection of the porta hepatis, a heparin bolus (30,000 U) was administered intravenously. The common bile duct, common hepatic artery, portal vein, infrahepatic inferior vena cava (IVC), and suprahepatic IVC were ligated prior to liver explant. No in situ aortic or portal flush was performed.

Donor Liver Preparation and Cannulation

The liver was topically cooled with ice in an organ basin on a sterile back table. The portal vein was cannulated with a 24 Fr cannula and flushed with 2 L of cold Normosol-R (FIG. 8, element B). The hepatic artery was cannulated with a 10-12 Fr cannula and flushed with 1.5 L of cold Normosol-R. The suprahepatic IVC was ligated and any remaining diaphragm was oversewn. The common bile duct was cannulated with an 8-12 Fr cannula and the infrahepatic IVC was cannulated with a 36-40 Fr single-stage, venous drainage cannula. A tissue specimen was collected from a randomly selected lobe of the liver for baseline histologic analysis.

Host Preparation and Cannulation

Host swine (n=4) underwent sedation, anesthetic induction, and preoperative preparation in the same fashion as donor swine. All hosts underwent endotracheal intubation and were continuously ventilated throughout the duration of the study. An auricular arterial line was placed for hemodynamic monitoring and periodic blood sampling. For anesthetic maintenance, inhaled isoflurane (1-3%) and fentanyl (0.03-0.1 mg/kg/hr) were supplemented with ketamine (5-15 mg/kg/hr) and midazolam (0.1-0.3 mg/kg/hr) as needed to maintain an appropriate plane of anesthesia throughout the experiment. Prior to skin incision, antibiotics (cefazolin, 20 mg/kg; enrofloxacin, 5 mg/kg) and immunosuppression (tacrolimus, 5 mg; mycophenolate mofetil, 500 mg; methylprednisolone, 1 g) were administered. Open cystostomy and bladder catheterization were performed for urine output monitoring. Exposure of the left and right internal jugular veins (IJV) was accomplished via bilateral cut-downs (FIG. 8, element C). A heparin bolus (30,000 U) was administered. The right IJV was used for drainage and cannulated with a 19 Fr cannula. The left IJV was used for venous return and cannulated with a 17 Fr cannula. A 12-14 Fr cannula was placed in the common femoral artery via open cutdown (FIG. 8, element D). Immediately following cannulae placement, extracorporeal support was initiated.

Cross-Circulation and Extracorporeal Liver Support

The XC circuit was primed with Normosol-R and donor blood. One gram of methylprednisolone and 1 gram of calcium chloride were administered. The circuit was connected to the venous and arterial cannulas and extracorporeal, veno-arterial-venous (V-AV), blood flow was initiated without initial inclusion of the extracorporeal liver. After confirming cannula site hemostasis and recipient hemodynamic stability on extracorporeal life support, the circuit was then clamped, briefly pausing extracorporeal blood flow. The relevant liver loop portions of the circuit were divided, and the portal vein, hepatic artery, and IVC cannulas were connected to appropriate inflow and outflow circuit components, thus splicing the extracorporeal liver into the circuit (FIG. 8, element A). Clamps were removed from hepatic inflow tubing, marking the start of veno-arterial-venous (V-AV) cross-circulation. After initial reperfusion, circuit flows were titrated to target 0.3 to 0.4 liters per minute (LPM) to the hepatic artery and 0.65 to 0.8 LPM to the portal vein, similar to ranges reported in prior liver NMP studies. In addition, flows were adjusted to achieve 1 LPM of arterial return to the host for V-A ECMO support. The height of the liver relative to the host was adjusted to target portal venous pressure <15 mmHg and hepatic venous pressure gradient (HVPG)<10 mmHg.

Cross-circulation blood flow, organ inflow and outflow pressures, and host hemodynamics were continuously monitored. Circuit and host temperature were maintained at 37° C. using a water heater and the oxygenator's water jacket. The extracorporeal liver was placed in an organ basin and covered with an isolation bag to prevent tissue desiccation and minimize insensible fluid loss. A drop sucker was placed underneath the liver, suction was applied with a roller pump, and connected to a cardiotomy reservoir to salvage and recirculate any blood loss or ascites volume. After 12 hours of cross-circulation, extracorporeal liver perfusion was discontinued, and the liver was flushed with 2 L of Normosol-R. The host animals were euthanized with sodium pentobarbital (125 mg/kg, IV)

Blood Collection and Analyses

Arterial blood samples were collected from the host's auricular line for blood gas and biochemical analysis prior to cross-circulation, immediately after start of cross-circulation, and every 6 hours thereafter. Blood samples were also collected from the circuit at pre- and post-extracorporeal liver access ports every 6 hours; metabolic parameters such as oxygen consumption and lactate clearance were derived from pre- and post-extracorporeal liver samples (calculation described below in Supplementary Methods). Blood gas analysis was performed using a point-of-care blood analysis system (epoc; Heska). Routine complete blood count and biochemical analyses were performed.

Bile Collection and Analyses

Bile was passively collected from the common bile duct via an 8-12 Fr cannula. Volume of bile production and bile pH (Orion Star, Thermo Scientific) were measured every 6 hours. Bile acids were measured by liquid chromatography-mass spectrometry.

Tissue Collection and Analyses

Baseline tissue specimens were collected from a randomly selected lobe of the liver prior to cross-circulation. Terminal tissue specimens were collected from a randomly selected region of the extracorporeal donor liver after 12 hours of cross-circulation. Donor hepatic artery, portal vein, and bile duct tissues were also collected at 12 hours. Necropsy was performed, and tissue specimens from the host's liver, spleen, kidney, lung, and lymph nodes were also collected. Tissue was fixed in 10% non-basic formalin for 48 hours, paraffin embedded, cut in 5 μm sections, and stained with Hematoxylin and Eosin (H&E), Gomori's Trichrome, and Periodic Acid-Schiff (PAS) stains. Brightfield microscopy was performed (Axioskop 40, Zeiss) and digital images obtained (Axiocam 305, Zeiss). Pathologic review was performed by an experienced gastrointestinal and liver pathologist. Injury scoring of hepatic parenchymal tissue was performed with blinded histopathologic assessment done with 4 technical replicates for each experiment at 0 h and 12 h timepoints. As shown in Table 1, assessment criteria included quantification of sinusoidal dilation, congestion, hepatocellular necrosis, fibrosis, vacuolation/steatosis, neutrophilic infiltration, and lymphocytic infiltration.

TABLE 1
Evaluation rubric of liver injury score.

| Injury score | 0 | 1 | 2 | 3 | 4 |
|---|---|---|---|---|---|
| Vacuolation/steatosis | <5% | 5-33% | 34-66% | >66% | |
| Sinusoidal dilation | None | Rare/focal | Patchy, <50% | Patchy, >50% | |
| | | | of the slide | of the slide | |
| Congestion | None | Rare/focal | Patchy, <50% | Patchy, >50% | |
| | | | of the slide | of the slide | |
| Hepatocellular | None | Rare/focal | <33% of the | 33-66% of the | >66% of the slide |
| necrosis | | dropout | slide | slide (bridging | (extensive, severe, |
| | | | | necrosis) | diffuse, panlobular |
| | | | | | necrosis) |
| Fibrosis | None | Portal | Periportal | Bridging | Cirrhosis |
| Neutrophils | <10 | 10-50 | 50-100 | >100 | |
| Lymphocytic | 0 | 1 | 2 | 3 | 4 |
| infiltration/inflammation | | | | | |
| Lymphocytes* | None | Minimal | Mild | Moderate | severe |

*assessment based on the Batts-Ludwig classification system.

Data Analysis

No data were excluded from analysis. Two-tailed, paired student's t-tests and one-way ANOVA with repeated measures (with Tukey's post-hoc analysis) were performed using statistical analysis software (Prism 9.0.0; Graph Pad) and p<0.05 was considered statistically significant. Continuous variables are summarized as means±standard error of the mean (SEM).

Results

These results show the feasibility of the extracorporeal organ support system in maintaining the structure, viability, and function of extracorporeal livers for 12 hours.

Extracorporeal Circuit Stability

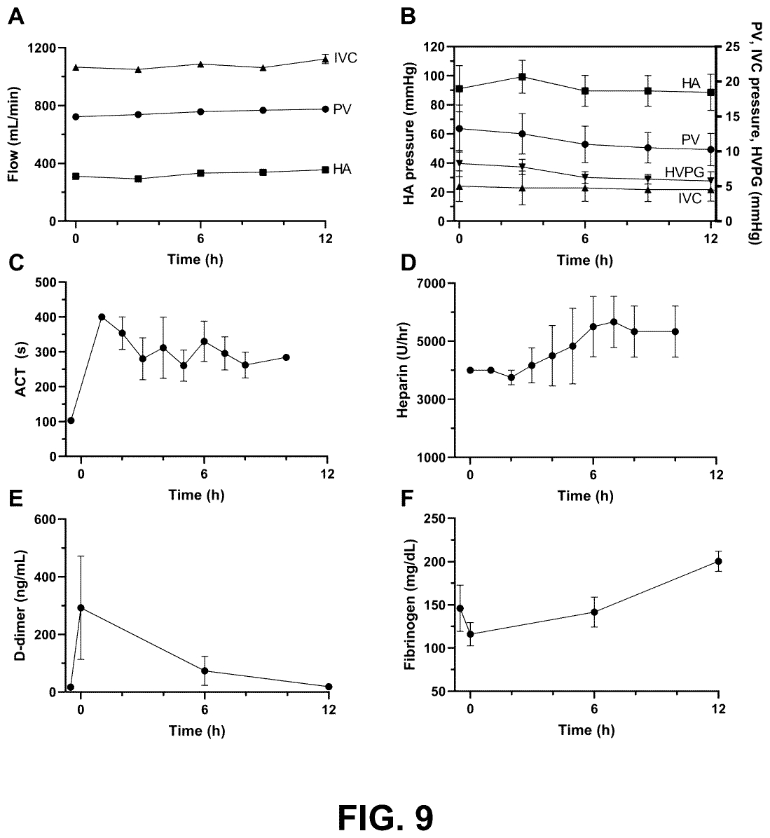

Extracorporeal circuit parameters were maintained within target liver-protective ranges throughout extracorporeal support, with target V-A ECMO (via femoral arterial return) flow 0.9-1.1 L/min. Hepatic artery flow was maintained at 0.33±0.02 L/min (0 hour, 0.31±0.02 L/min; 12 hour 0.36±0.02 L/min), portal venous flow was maintained at 0.75±0.02 L/min (0 hour, 0.72±0.01 L/min; 12 hour 0.77±0.01 L/min), and total caval flow was maintained at 1.08±0.02 L/min (0 hour, 1.06±0.02 L/min; 12 hour 1.13±0.03 L/min) (FIG. 9, element A). Hepatic artery pressures remained below 120 mmHg. HVPG, the difference between portal and caval pressures, was maintained at target <10 mmHg (FIG. 9, element B). Activated clotting time (ACT) was targeted to 200 to 300 seconds with a heparin infusion (FIGS. 9, element C and 9, element D). D-dimer peaked (FIG. 9, element E) while fibrinogen nadired (FIG. 9, element F) at onset of cross-circulation, but both subsequently normalized.

Maintenance of physiologic hemodynamic parameters is critical for optimizing oxygen and nutrient delivery, as well as limiting vascular stress and hepatocellular injury. Hepatic arterial pressure and flow remained within physiologic ranges, which reflects intact autoregulatory functions of the myogenic response as well as the hepatic arterial buffer response. Portal pressure, flow, and HPVG were also maintained within physiologic range, which prevents hepatic congestion and centrilobular necrosis.

Host Swine Safety and Stability

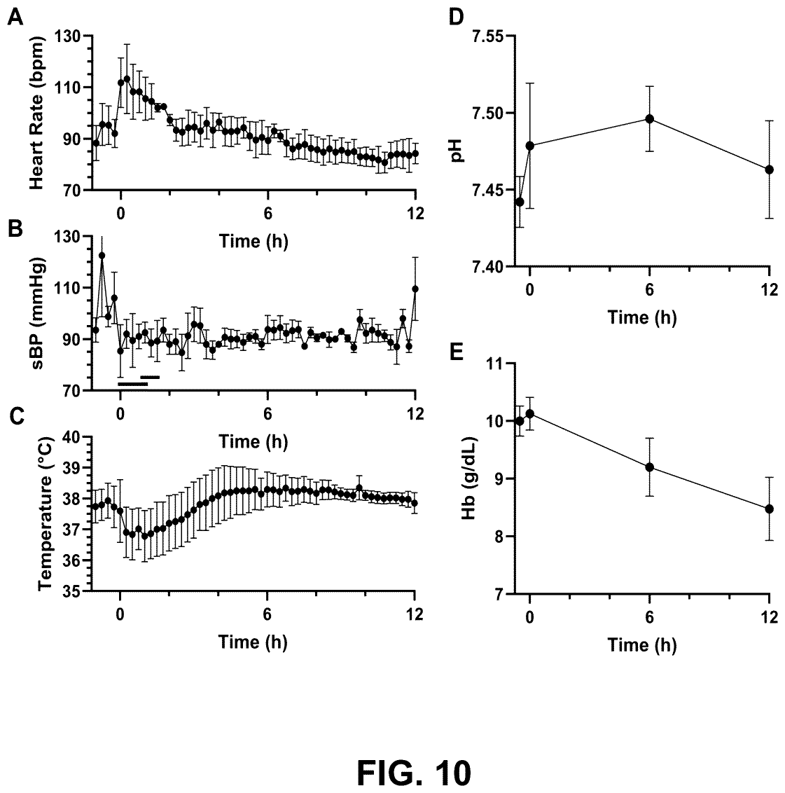

Safety and stability were assessed by monitoring of host swine vitals and hemodynamic parameters—which were maintained within normal ranges after transient instability with initiation of cross-circulation (FIGS. 7A and 7B, mean heart rate, 91±2 bpm and mean systolic pressure, 92±2 mm Hg)—and by blood gas analysis (FIG. 10, element D, mean pH, 7.47±0.03). Hemoglobin concentration did not change significantly over the 12 hours of XC (FIG. 10, element E, pre-XC, 10.0±0.3 g/dL; hour 12, 8.5±0.5 g/dL; p=0.12). Additional blood gas, blood counts, serum chemistry, and serum coagulation studies are reported in Table 2. Terminal histologic evaluation of various host tissue demonstrated no major abnormalities.

TABLE 2
Safety and stability of host swine during 12 hours of cross-circulation
support: analysis of host biochemistry, coagulation, and electrolytes.

| | Timepoint (h) | | | |
|---|---|---|---|---|
| Parameter | Pre-XC | 0 | 6 | 12 |
| Vitals | | | | |
| Heart rate (bpm) | 92 ± 7 | 110 ± 5 | 90 ± 4 | 84 ± 5 |
| Systolic BP (mmHg) | 105 ± 13 | 90 ± 7 | 94 ± 4 | 95 ± 8 |
| Temperature (° C.) | 37.8 ± 0.5 | 37.1 ± 0.7 | 38.3 ± 0.5 | 38.0 ± 0.2 |
| Arterial blood gas | | | | |
| pH | 7.44 ± 0.02 | 7.48 ± 0.04 | 7.50 ± 0.02 | 7.46 ± 0.03 |
| O₂ tension (mmHg) | 358 ± 61 | 485 ± 59 | 485 ± 51 | 389 ± 89 |
| CO₂ tension (mmHg) | 51 ± 4 | 46 ± 3 | 46 ± 3 | 50 ± 4 |
| Bicarbonate (mmol/L) | 34 ± 2 | 34 ± 1 | 35 ± 1 | 35 ± 1 |
| Lactate (mmol/L) | 1.7 ± 0.2 | 3.1 ± 0.9 | 1.2 ± 0.1 | 0.9 ± 0.1 |
| Glucose (mg/dL) | 134 ± 14 | 152 ± 27 | 190 ± 8 | 240 ± 18 |
| Blood counts | | | | |
| WBC (10⁹/L) | 18.3 ± 2.3 | 12.6 ± 1.6 | 19.0 ± 2.5 | 15.9 ± 0.9 |
| % Neutrophil | 58 ± 8 | 57 ± 6 | 81 ± 2 | 76 ± 1 |
| % Lymphocytes | 40 ± 7 | 40 ± 6 | 17 ± 2 | 19 ± 2 |
| Platelets (10⁹/L) | 388 ± 39 | 287 ± 66 | 258 ± 67 | 263 ± 59 |
| Hemoglobin (g/dL) | 10.0 ± 0.3 | 10.1 ± 0.3 | 9.2 ± 0.5 | 8.5 ± 0.5 |
| Chemistries | | | | |
| Sodium (mmol/L) | 138.5 ± 1.9 | 138.3 ± 2.4 | 140.2 ± 1.7 | 141.5 ± 1.6 |
| Potassium (mmol/L) | 4.34 ± 0.04 | 4.2 ± 0.1 | 4.8 ± 0.2 | 4.7 ± 0.1 |
| Calcium (mg/dL) | 9.8 ± 0.6 | 9.8 ± 0.2 | 9.6 ± 0.2 | 9.9 ± 0.2 |
| BUN (mg/dL) | 11.5 ± 1.3 | 13.5 ± 1.7 | 24.5 ± 2.4 | 33 ± 3.5 |
| Creatine (mg/dL) | 1.0 ± 0.1 | 1.0 ± 0.1 | 1.1 ± 0.3 | 1.0 ± 0.2 |
| Albumin (g/dL) | 3.2 ± 0.2 | 3.0 ± 0.2 | 3.0 ± 0.1 | 3.2 ± 0.1 |
| AST (U/L) | 16 ± 6 | 163 ± 54 | 163 ± 33 | 173 ± 50 |
| ALT (U/L) | 24 ± 6 | 37 ± 3 | 38 ± 2 | 38 ± 4 |
| ALP (U/L) | 115 ± 16 | 135 ± 8 | 120 ± 3 | 136 ± 6 |
| LDH (U/L) | 315 ± 58 | 370 ± 56 | 432 ± 24 | 630 ± 133 |
| Coagulation studies | | | | |
| PT (s) | 10.2 ± 1.0 | 15 ± 4.1 | 10.7 ± 0.4 | 9.7 ± 0.2 |
| D-dimer (ng/mL) | 17 ± 1 | 293 ± 179 | 74 ± 50 | 19 ± 3 |
| Fibrinogen (mg/dL) | 146 ± 27 | 116 ± 13 | 142 ± 17 | 200 ± 12 |

Values are presented as mean ± standard error of the mean. BP, Blood pressure; WBC, white blood cells; Hgb, hemoglobin; Hct, hematocrit; BUN, blood urea nitrogen; AST, aspartate transaminase; ALT, alanine transaminase; PTT, partial thromboplastin time; PT, prothrombin time.

The XC concurrently provided approximately 1 L/min of V-A ECMO hemodynamic support to the swine host throughout the experiment. Outside of brief periods of hypotension at the onset of cross circulation in 2 of 4 hosts, readily treated with norepinephrine, there were no other episodes of host instability throughout cross-circulation. This initial instability was likely secondary to flushing of metabolites and cold perfusate from the extracorporeal liver, combined with rapid intravascular volume shifts as the extracorporeal liver is perfused. This observation parallels the physiologic response seen clinically in liver transplant recipients experiencing post-reperfusion syndrome, and the use of the V-A component suggests an opportunity to minimize the impact of reperfusion-associated instability.

Gross and Histologic Assessment

Gross imaging of extracorporeal livers demonstrated normal appearance of hepatic surfaces, maintenance of global hepatic structure, and uniform perfusion. Histologic evaluation demonstrated preservation of hepatic lobular and sinusoidal structural integrity with no evidence of hepatocellular necrosis or substantial periportal inflammation after 12 hours of extracorporeal liver perfusion. Trichrome staining revealed maintenance of normal appearing lobular architecture and portal triad structures. No substantial glycogen accumulation was observed on histologic examination with PAS special stain. Histopathologic liver injury scoring demonstrated a statistically significant decrease in sinusoidal dilation (−0.63 points; 95% CI: −1.14 to −0.12 points; p=0.020) and composite acute injury score (−0.75 points; 95% CI: −1.41 to −0.09 points; p=0.029) between 0-hour and 12-hour timepoints. Hepatocellular congestion and necrosis remained low and without significant change. No steatosis or fibrosis was observed in any livers at either timepoint. Lymphocytic infiltration was significantly higher at 12 hours than at 0 hours.

TABLE 3
Histopathologic evaluation of additional host and donor tissues.

| Tissue | Summary of pathologic commentary |
|---|---|
| Host tissue | |
| Liver parenchyma | Mild sinusoidal lymphocytic inflammation |
| Kidney | Normal |
| Lung | Mild interstitial chronic inflammation |
| Lymph node | Near normal lymph nodes with focal acute |
| | inflammation |
| Spleen | Normal |
| Thymus | Normal |
| Donor tissue | |
| Bile duct | Mild chronic inflammation, focal epithelial |
| | denudation |
| Hepatic artery | Minimal acute inflammation |
| Portal vein | Normal |

Functional and Metabolic Assessment of the Extracorporeal Liver

Liver weight remained stable over the course of cross-circulation Oxygen consumption, calculated based on the Fick principle, was maintained and did not demonstrate a statistically significant change (hour 0, 1.7±0.6 mL/min/100 g; hour 12, 2.4±0.2 mL min/100 g; p=0.22). Percent lactate clearance by the extracorporeal liver increased (hour 0, 24±13%; hour 12, 48±7%; 95% CI: 0.02 to 48.5%; p=0.0499). Perfusate blood urea nitrogen (BUN) positively correlated with time (p=0.004). The extracorporeal liver demonstrated stable bile production with alkaline bile composition throughout cross-circulation. Species of the most abundant bile acids were unaltered.

Markers of Hepatocellular Injury

AST levels increased at reperfusion, but thereafter remained stable throughout XC (pre-XC, 15.8±5.5 U/L; 0 h, 163±54 U/L; p=0.13). ALT levels likewise increased at reperfusion and stabilized (FIG. 6B; pre-XC, 24.3±6.4 U/L; 0 h, 37.3±3.2 U/L; p=0.17). Alkaline phosphatase and lactate dehydrogenase levels remained stable.

### Example 2

Described herein is the use of xenogeneic cross-circulation (XCC) for the support of explanted human livers for 24 hours. It was demonstrated that XCC enables the physiologic support of explanted human livers for 24 hours. XCC has potential application as a translational research platform and clinical biotechnology for organ salvage and recovery.

Methods

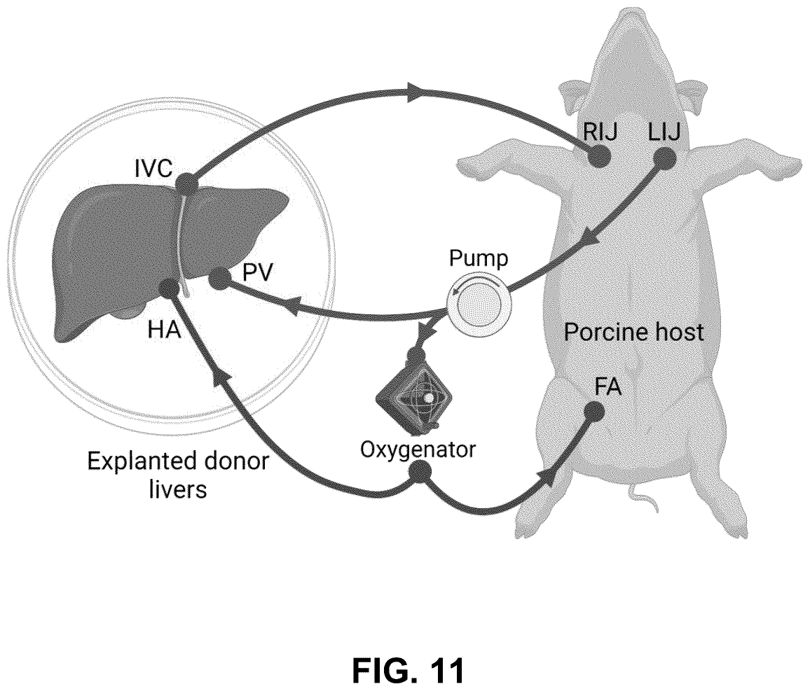

Human donor livers (n=2) declined for clinical transplantation were procured and placed on normothermic, veno-arteriovenous XCC with anesthetized, immunosuppressed, complement depleted porcine hosts (FIG. 11). Longitudinal analyses of extracorporeal livers and porcine hosts were performed over 24 hours of XCC.

Results

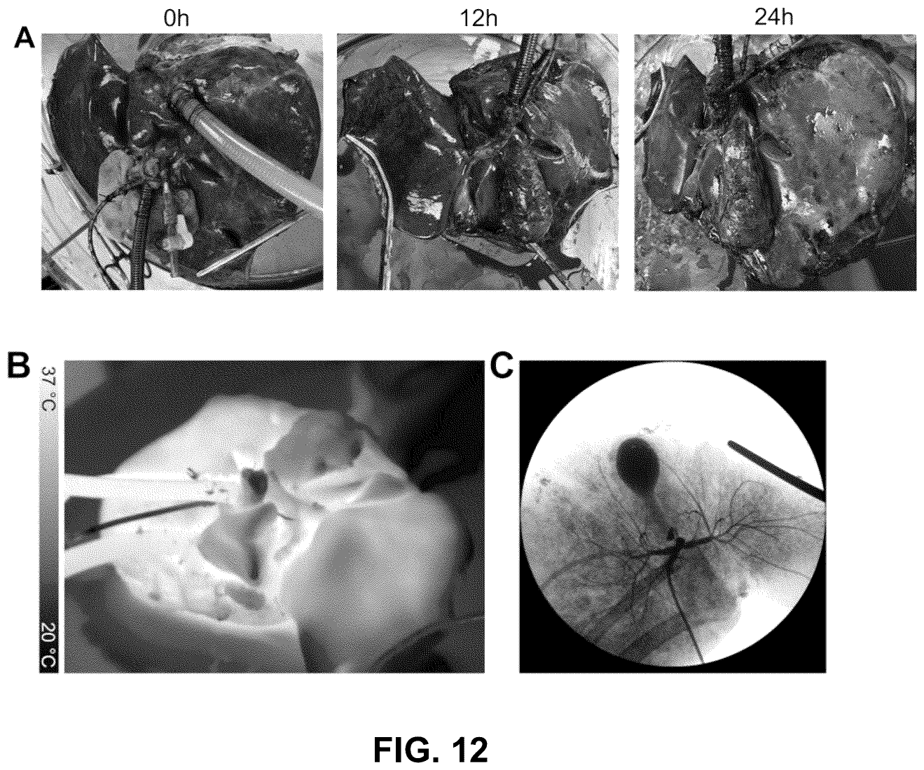

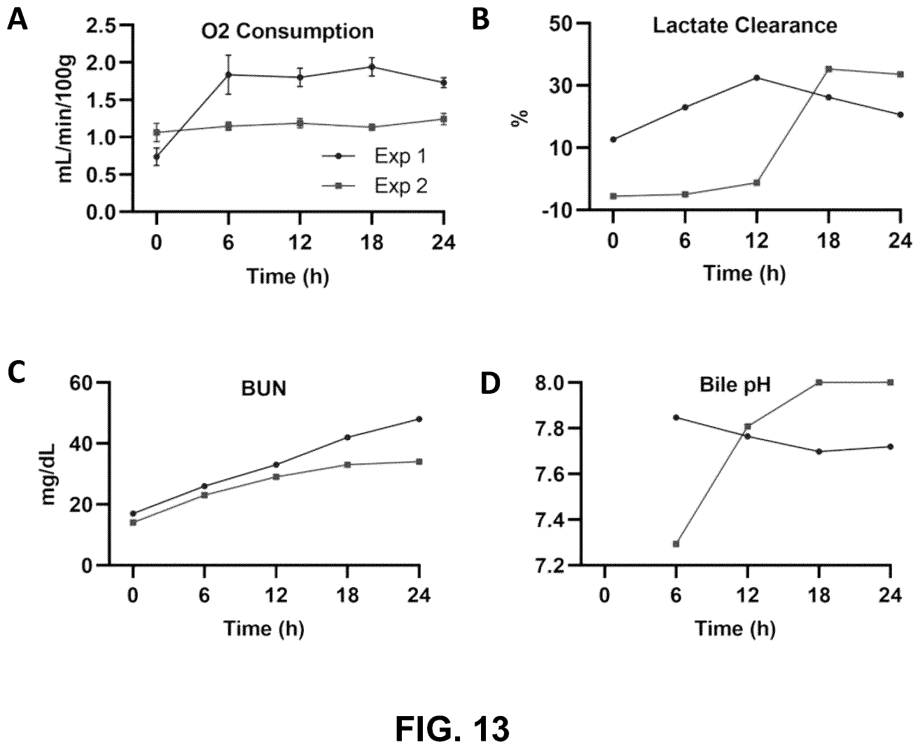

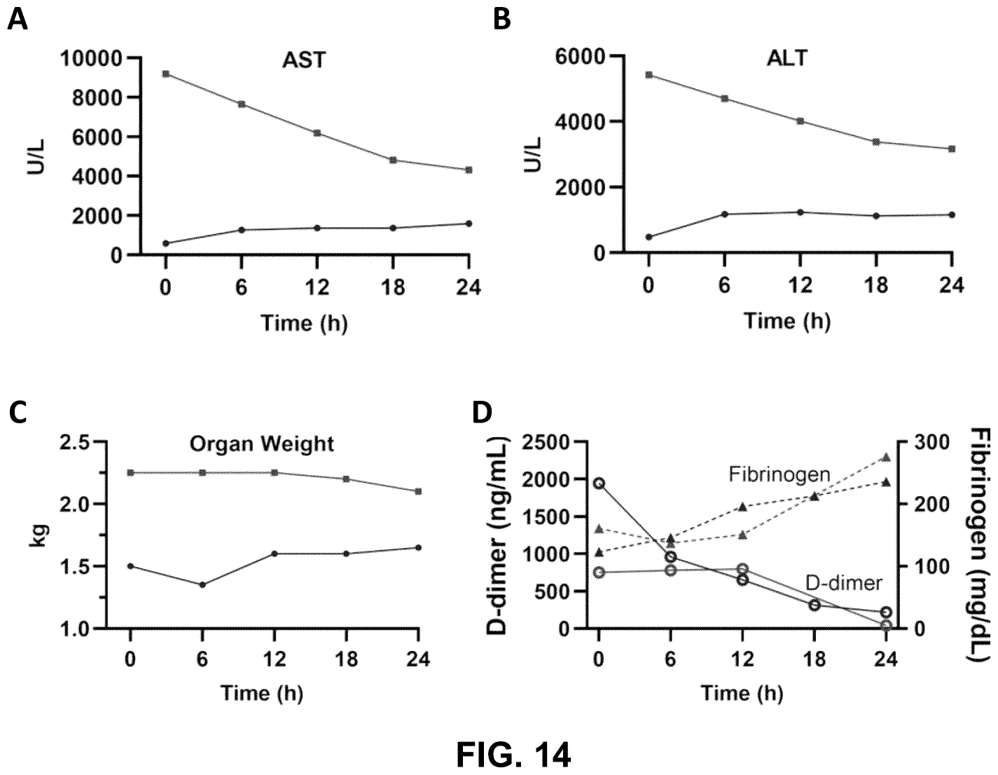

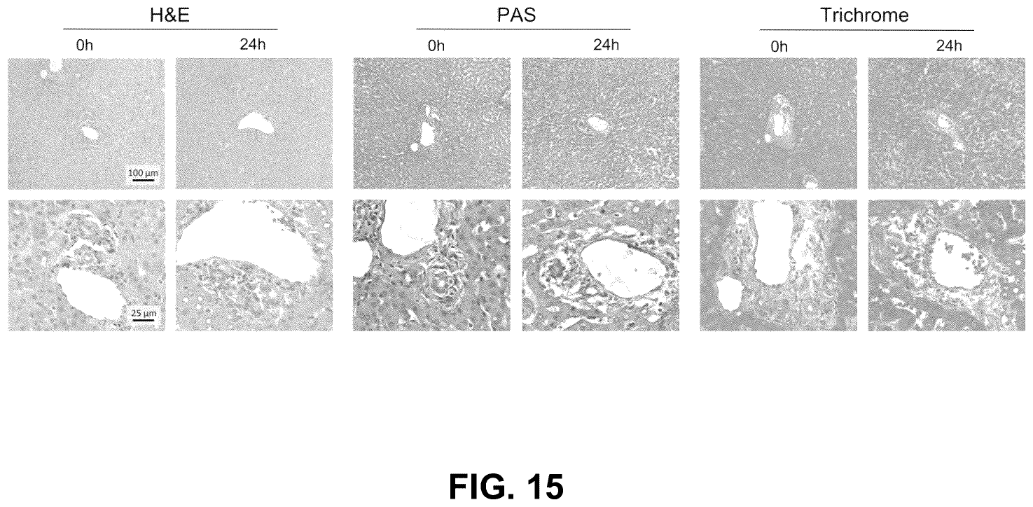

Throughout 24 hours of support, extracorporeal livers maintained gross architecture, normothermic perfusion, and biliary integrity (FIG. 12, elements A, B, and C). Functionally, the liver demonstrated stable or improved oxygen consumption, lactate clearance, protein metabolism, and alkaline bile composition (FIG. 13, elements A, B, C, and D). Liver enzymes increased upon reperfusion, but decreased or remained stable throughout cross-circulation (FIG. 14, elements A and B). Organ weight remained stable and fibrinolytic markers improved over the course of support (FIG. 14, elements C and D). There was no major histologic evidence of hepatocellular injury (FIG. 15). Circuit parameters remained physiologic during XCC (hepatic artery flow 233±55 mL/min; hepatic artery pressure 89±45 mmHg; portal venous flow 510±120 mL/min; portal venous pressure 10±3 mmHg; portal venous O2 saturation 64±15%).

From the above description, those skilled in the art will perceive improvements, changes, and modifications. Such improvements, changes and modifications are within the skill of one in the art and are intended to be covered by the appended claims.

The following is claimed:

1. A system comprising:
an organ chamber configured to hold an extracorporeal organ; and
a cross-circulation circuit configured to connect the extracorporeal organ and a host organism to maintain the extracorporeal organ by perfusing veno-arterial venous (V-AV) blood through the extracorporeal organ and the host organism, wherein physiologic stability of the host organism is maintained.

2. The system of claim 1, wherein the extracorporeal organ is at least one of a liver, a lung, a kidney, a heart, a limb, skin, or a tissue substrate.

3. The system of claim 1, wherein the extracorporeal organ is a liver, and
wherein a portal vein of the liver, a hepatic artery of the liver, and an infrahepatic inferior vena cava are cannulated to allow perfusion of the V-AV blood through the liver and the host organism.

4. The system of claim 3, wherein the infrahepatic inferior vena cava is connected to a peripheral or central vein of the host organism, and another peripheral or central vein of the host organism is connected with the portal vein and the hepatic artery of the liver through a pump and an oxygenator, and also to a peripheral or central artery of the host organism.

5. The system of claim 1, further comprising a heater to keep the extracorporeal organ, the host organism, and the cross-circulation circuit at a constant temperature from 10 degrees Celsius to 50 degrees Celsius.

6. The system of claim 1, wherein the organ chamber is at a variable height based on a height of the host organism.

7. The system of claim 1, wherein the variable height of the organ chamber keeps the organ and the host organism at a near physiologic pressure.

8. The system of claim 1, wherein the system further comprises:
a plurality of sensors configured to detect changes in at least one parameter of the cross-circulation circuit, the organ and/or the host organism; and
a monitoring device configured to monitor changes and alerting a medical professional when changes are outside a pre-determined threshold.

9. The system of claim 8, wherein the at least one parameter includes at least one of a blood flow rate, a cross circulation blood flow, an organ inflow pressure, an organ outflow pressure, a host hemodynamics value, a circuit temperature, and a host temperature.

10. The system of claim 8, further comprising:
a pump configured to pump blood from at least one vein of the host organism to at least one artery of the host organism;
an oxygenator configured to oxygenate the blood before the blood enters the at least one artery of the host organism; and
a heater,
wherein the monitoring device controls at least one of the oxygenator, the pump, and the heater in response to detecting changes in the at least one parameter outside the pre-determined threshold to return the at least one parameter to within the predetermined threshold.

11. The system of claim 1, wherein the cross-circulation circuit further comprises:
a pump configured to be connected to at least one vein of the host organism, at least one vein or artery of the extracorporeal organ, and at least one artery of the host organism via at least one tube.

12. The system of claim 11, wherein the pump is configured to use negative pressure to pull blood from the at least one vein of the host organism and to use positive pressure to push the blood into the at least one artery of the host organism and the at least one vein or artery of the extracorporeal organ, wherein a flow regulator is configured to control the rate of blood pumped into the at least one artery of the host and the rate of blood pumped into the at least one vein or artery of the extracorporeal organ.

13. The system of claim 11, wherein the cross-circulation circuit further comprises an oxygenator configured to maintain a physiologic level of oxygen in the blood perfusing the extracorporeal organ and the host organism.

14. A method, comprising:
maintaining viability of an extracorporeal organ, wherein the extracorporeal organ is located in an organ chamber;
cannulating at least one vein and one artery in the extracorporeal organ;
cannulating at least two veins of a host organism, and an artery of the host organism;
establishing a cross-circulation circuit by connecting:
one of the at least two veins of the host organism to be in fluid communication with the artery of the host organism and to be in fluid communication with the at least one artery in the extracorporeal organ, and
the at least one vein of the extracorporeal organ to be in fluid communication with another of the at least two veins of the host organism; and
perfusing veno-arterial venous (V-AV) blood through the extracorporeal organ and the host organism, wherein physiologic stability of the host organism is maintained.

15. The method of claim 14, wherein cannulating the at least two veins of the host organism further comprises cannulating an internal jugular vein of a host organism and cannulating a contralateral internal jugular vein of the host organism.

16. The method of claim 15, wherein establishing the cross-circulation circuit further comprises connecting the at least one vein of the extracorporeal organ with the internal jugular vein of the host organism and connecting the artery of the host organism with the contralateral internal jugular vein of the host organism.

17. The method of claim 16, wherein the extracorporeal organ is a liver and cannulating the at least one vein and one artery in the extracorporeal organ further comprises cannulating the liver's hepatic artery, inferior vena cava, and portal vein.

18. The method of claim 17, wherein establishing the cross-circulation circuit further comprises:
connecting the internal jugular vein to the artery of the host organism and to the portal vein of the liver and the hepatic artery of the liver; and
connecting the infrahepatic inferior vena cava to the contralateral internal jugular vein of the host organism,
wherein blood flows from the internal jugular vein of the host organism to the artery of the host organism and the hepatic artery and portal vein of the liver, and blood flows from the liver through the inferior vena cava to the contralateral internal jugular vein of the host organism.

19. The method of claim 14, further comprising maintaining the extracorporeal organ, the host organism, and the cross-circulation circuit at a constant temperature from 10 degrees Celsius to 50 degrees Celsius.

20. The method of claim 14, further comprising:
detecting, by a plurality of sensors, changes in at least one parameter of the cross-circulation circuit, the organ, and the host organism;
monitoring, by a monitoring device, the changes in the at least one parameter of the cross-circulation circuit, the host organism, and the organ, wherein the at least one parameter includes at least one of blood flow rates, cross circulation blood flow, organ inflow pressure, organ outflow pressure, host hemodynamics, circuit temperature, and host temperature; and
alerting, by a display of the monitoring device, a medical professional when changes are outside a pre-determined threshold.

21. The method of claim 14, further comprising regulating the flow rate of blood from the one of the at least two veins of the host organism to the artery of the host organism and to the at least one artery in the extracorporeal organ.
